(19) United States
(12) Patent Application Publication
(10) Pub. No.: US 2025/0366711 A1
(43) Pub. Date: Dec. 4, 2025

(54) SYSTEMS AND METHODS FOR IDENTIFYING EYE GAZE PATTERN WITH RESPECT TO VISUAL STIMULUS

(71) Applicant: TREE BEAR LIMITED, Hong Kong (HK)

(72) Inventor: Wai Lun TSE, Hong Kong (HK)

(21) Appl. No.: 19/222,256

(22) Filed: May 29, 2025

Related U.S. Application Data
(60) Provisional application No. 63/653,255, filed on May 30, 2024.

(51) Int. Cl. A61B 3/032; G06F 3/01; G06T 7/80; G06V 40/18; G16H 30/20

(52) U.S. Cl. CPC A61B 3/032; G06F 3/013; G06T 7/80; G06V 40/18; G16H 30/20

(57) ABSTRACT

Methods and apparatuses for pre-screening, detecting or monitoring developmental, cognitive, social, or mental disabilities or abilities. The simple, low-cost, quick and highly deployable methods and apparatuses employ artificial intelligence (AI) to analyze at least an individual's gaze pattern in response to a visual stimulus. The method comprises detecting a head and an eye region of the individual, obtaining a head pose of the individual, comparing the obtained head pose with a predetermined threshold range, obtaining one or more eye gaze parameters with regard to individual's eye gaze towards at least one point-of-interest in the visual stimulus using the selected eye gaze direction based on the head pose comparison step.

### CROSS REFERENCE TO RELATED APPLICATIONS

This application claims the benefit of U.S. Provisional Patent Application No. 63/653,255, filed May 30, 2024, the disclosure of which is hereby incorporated by reference.

### FIELD OF INVENTION

The present invention generally relates to methods and apparatuses for tracking eye gaze. Particularly, although not exclusively, the present invention relates to assessing developmental, cognitive, social, or mental disability or ability of an individual via eye gaze tracking and identifying eye gaze with respect to visual stimulus. The present invention also relates to gamification via eye gaze tracking and identifying eye gaze with respect to visual stimulus.

### BACKGROUND

A developmental, cognitive, social, or mental disability, for example, attention-deficit/hyperactivity disorder (ADHD) and autism spectrum disorders (ASD) present challenges for affected children from a young age, contributing to a concerning rise in their prevalence. These disorders also significantly impact caregivers and parents, who experience heightened stress, poor mental health, and internalized stigma. Diagnostic methods for these conditions vary widely. For example, some diagnostic methods require healthcare providers to conduct manual screenings and assessments for each individual. To obtain insights from the screenings and assessments, the data obtained are required to be further processed and validated manually. These diagnostic methods are expensive and time consuming. Some other diagnostic methods require special equipment in a laboratory. While special equipment is expensive and not easily accessible, its bulkiness often renders it unsuitable for children. It is usually complicated and requires a number of calibrations prior to the diagnosis. The bulkiness and calibration process may cause anxiety and stress in children, potentially impacting the accuracy of diagnostic results. In addition, existing diagnostic methods often lack quantitativeness and heavily rely on subjective judgement, which can lead to inconsistency and variability in diagnoses. As a result, it may cause delays in treatment and misdiagnoses for patients. Furthermore, healthcare providers, such as pediatricians, lack adequate tools and data enabled by latest technology to monitor progress, particularly in early childhood.

### SUMMARY

In view of the foregoing, it is an objective of the present invention to provide simple, low-cost, quick and highly deployable methods and apparatuses for pre-screening, detecting or monitoring developmental, cognitive, social, or mental disabilities or abilities. To achieve the objective, it is an aspect of the present invention to employ artificial intelligence (AI) to analyze at least one predetermined behavior of an individual in real time. Particularly, the present invention uses vision-AI to identify and analyze at least one predetermined part of the body of the individual and analyze its respective behavior. The at least one predetermined part of the body includes, but not limited to, the eyes and the head of the individual and the at least one behavior includes, but not limited to the eyes gaze, eyelids and the head pose of the individual.

The employment of AI in the present invention enhances both objective and quantitative measurements in pre-screening, detecting and monitoring developmental, cognitive, social, or mental disabilities or abilities of the individual due to its ability to process data in detail. It allows the measurement of metrics with a high level of precision and accuracy. The objective and quantitative measurements together with the AI analysis improve the consistency in pre-screenings, detections and monitors across individuals.

In addition, in some embodiments, the present invention uses a tablet computer to pre-screen, detect and/or monitor developmental, cognitive, social, or mental disabilities or abilities of individuals in real time. As a result, the pre-screening, detection and monitor process may be performed in an ordinary room. Familiarity with the tools and environment helps reduce anxiety and stress during the process (especially in children), which enhance the accuracy of the results.

Further to the foregoing aspects, the pre-screening, detection and monitoring methods and apparatuses of the present invention may be easily accessible, leading to opportunities for early intervention. In another aspect, in some embodiments, the present invention may be used as an EdTech (education technology) tool to pre-screen, detect and/or monitor developmental, cognitive, social, or mental disabilities or abilities of children across their childhood, facilitating customization of education experience for each child based on his/her disability or ability. It greatly enhances the quality and efficiency in SEN education (special educational needs education). In yet another aspect, in some embodiments, the present invention may act as an EdTech tool which gamifies the learning process through body behavior analysis, including but not limited to, eye gaze analysis. Engagement and interaction experience may be improved.

In another aspect of the present invention, the methods and apparatuses of eye gaze tracking with respect to visual stimulus may be used to provide gamification experiences to individuals and have applications in and without the field of medical diagnosis. It includes other industries related to tracking eye gaze corresponding to a point-of-interest of visual stimulus. For example, it may gamify therapeutic tools and processes to enhance therapy efficiency or gamify advertisements in marketing to enhance customers' experience.

Of the many aspects of the invention, therefore, is a method for identifying an individual's gaze pattern in response to a visual stimulus comprising the steps of detecting a head and at least one eye region of the individual, obtaining a head pose of the individual, comparing the obtained head pose with a predetermined threshold range and if the obtained head pose falls within the threshold range, selecting an eye gaze direction of the individual associated with such head pose, obtaining one or more eye gaze parameters with regard to individual's eye gaze towards at least one point-of-interest in the visual stimulus using the selected eye gaze direction based on the head pose comparison step, wherein the one or more eye gaze parameters are (a) a marker of pre-screening, detecting or monitoring a developmental, cognitive, social, or mental disability or ability or (b) used to generate a gamification element.

In some embodiments, the method further comprises the step of defining a plurality of spatial region-of-interests in the visual stimulus wherein the spatial region-of-interests form a substantial continuous coverage over the entire visual stimulus, wherein an individual's eye gaze towards at least one point-of-interest in the visual stimulus is obtained by determining the individual's eye gaze towards a pre-assigned spatial region-of-interest corresponding to the respective point-of-interest, and wherein the one or more eye gaze parameters comprise a total gazing time for which the individual's eye gaze is towards the pre-assigned spatial region-of-interest.

In some embodiments, the method further comprises the step of obtaining one or more head pose parameters with regard to individual's head pose based on the head pose comparison step, wherein the one or more head pose parameters comprise a total time for which the individual's head pose exceeds the threshold, and wherein the one or more head pose parameters are (a) a marker of pre-screening, detecting or monitoring a developmental, cognitive, social, or mental disability or ability or (b) used to generate a gamification element.

In another aspect, the present invention is a non-transitory computer-readable storage medium storing one or more programs, the one or more programs comprising instructions, which when executed by one or more processors of an electronic device, cause the electronic device to identify an individual's gaze pattern in response to a visual stimulus comprising the steps of detecting a head and at least one eye region of the individual, obtaining a head pose of the individual, comparing the obtained head pose with a predetermined threshold range and if the obtained head pose falls within the threshold range, selecting an eye gaze direction of the individual associated with such head pose, obtaining one or more eye gaze parameters with regard to individual's eye gaze towards at least one point-of-interest in the visual stimulus using the selected eye gaze direction based on the head pose comparison step, wherein the one or more eye gaze parameters are (a) a marker of pre-screening, detecting or monitoring a developmental, cognitive, social, or mental disability or ability or (b) used to generate a gamification element.

In yet another aspect, an eye gaze tracking system for identifying an individual's gaze pattern in response to a visual stimulus comprising at least one image capturing device to capture a head and at least one eye of the individual, at least one display unit to display the visual stimulus to the individual, at least one processor to receive and process the captured images and/or video from the image capturing device, a head detection module to detect a head of the individual from the captured images and/or video, an eye detection module to detect at least one eye region of the individual from the captured images and/or video, an eye gaze detection module to determine an eye gaze direction of the individual from the detected eye region, a head pose estimation module to obtain an head pose and compare the obtained head pose with a predetermined threshold range and if the obtained head pose falls within the threshold range, selecting an eye gaze direction of the individual associated with such head pose; and a data fusion module to obtaining one or more eye gaze parameters with regard to individual's eye gaze towards at least one point-of-interest of the visual stimulus using the selected eye gaze direction based on the head pose comparison step, wherein the one or more eye gaze parameters is (a) a marker of pre-screening, detecting or monitoring a developmental, cognitive, social, or mental disability or ability or (b) used to generate a gamification element.

Additional aspects of the invention will be set forth in part in the description which follows and, in part, will be obvious from the description, or may be learned by practice of the invention.

### BRIEF DESCRIPTION OF DRAWINGS

Persons of ordinary skill in the art may appreciate that elements in the figures are illustrated for simplicity and clarity so not all connections and options have been shown. For example, common but well-understood elements that are useful or necessary in a commercially feasible embodiment may often not be depicted in order to facilitate a less obstructed view of these various embodiments of the present disclosure. It may be further appreciated that certain actions and/or steps may be described or depicted in a particular order of occurrence while those skilled in the art may understand that such specificity with respect to sequence is not actually required. It may also be understood that the terms and expressions used herein may be defined with respect to their corresponding respective areas of inquiry and study except where specific meanings have otherwise been set forth herein.

FIG. 1 is a schematic view of an eye gaze tracking system according to certain embodiments of the present invention.

FIG. 2 is a block diagram of an eye gaze racking system according to certain embodiments of the present invention.

FIG. 3 is an illustrative view of four spatial region-of-interests according to certain embodiments of the present invention.

FIG. 4A is an illustrative view of upper and lower spatial region-of-interests according to certain embodiments of the present invention. FIG. 4B is an illustrative view of left and right spatial region-of-interests according to certain embodiments of the present invention.

FIG. 5 is a schematic view of an eye gaze tracking system including a stand holder according to certain embodiments of the present invention.

FIG. 6 is a schematic view of an eye gaze tracking system including a holder according to certain embodiments of the present invention.

FIG. 7 shows an illustrative flowchart with computer-implemented functions for identifying eyes gaze of an individual corresponding to at least one spatial region-of-interest with respect to at least one point-of-interest in a visual stimulus shown to the individual according to certain embodiments of the present invention.

FIG. 8 shows an illustrative flowchart with computer-implemented functions for a response data analysis process according to certain embodiments of the present invention.

FIG. 9 shows an illustrative flowchart with computer-implemented functions for a response data analysis process according to other embodiments of the present invention.

FIG. 10 shows an illustrative screenshot exemplifying a representative report, according to one embodiment of the present invention.

### DETAILED DESCRIPTION

The present disclosure introduces systems and methods for tracking eye gaze and identifying eye gaze with respect to visual stimulus of an individual. The systems and methods gather data on visual stimuli preferences and may be configured to pre-screen, detect, and/or monitor various developmental, cognitive, social, or mental disabilities or abilities in an individual. Developmental, cognitive, social, or mental disabilities or abilities, includes but not limited to autism spectrum disorders (ASD), language delays, language levels, intellectual disabilities, traumatic brain injuries, attention-deficit/hyperactivity disorder (ADHD), PTSD, sports injuries, and dementia, cognitive function, and social development.

As used herein, pre-screening means determining whether an individual exhibits signs or characteristics indicative of a particular developmental, cognitive, social, or mental disability or ability. As used herein, detecting means identifying and evaluating the severity or extent of a particular developmental, cognitive, social, or mental disability or ability in an individual.

As used herein, “gamification” or “gamify” means the application of game-like elements, such as points, badges, leaderboards, and challenges, to a non-game environment. These elements are designed to increase user engagement, motivation, and encourage desired behaviors within the system. In particular, but not exclusively includes, comparing real-time values against an objective to motivate an individual to reach that objective. Gamification objectives may include predetermined metrics, predetermined eye gaze direction, or predetermined point-of-interest. This may include identifying how the current eye gaze is progressing compared to the predetermined eye gaze.

Artificial intelligence (AI), including machine learning, deep learning, and/or other artificial machine-driven logic, enables machines (e.g., computers, logic circuits, etc.) to use a model to process input data to generate an output based on patterns and/or associations previously learned by the model via a training process. For instance, the model may be trained with data to recognize patterns and/or associations and follow such patterns and/or associations when processing input data such that other input(s) result in output(s) consistent with the recognized patterns and/or associations.

In some embodiments, the individuals are children between ages of 2-6 years old (inclusive). In some embodiments, the individuals are toddlers and infants between ages of 6 months to 2 years old (inclusive). In some embodiments, the individuals are between ages of 7-16 years old (inclusive). In some embodiments, the individuals are under 21 years old. In some embodiments, the individuals are 21 years old or above.

The present disclosure also introduces methods and apparatuses of eye gaze tracking with respect to visual stimulus which can be used to provide gamification experiences to individuals. For example, it can gamify therapeutic tools and processes to enhance therapy efficiency.

Embodiments may now be described more fully with reference to the accompanying drawings, which form a part hereof, and which show, by way of illustration, specific exemplary embodiments which may be practiced. These illustrations and exemplary embodiments may be presented with the understanding that the present disclosure is an exemplification of the principles of one or more embodiments and may not be intended to limit any one of the embodiments illustrated. Embodiments may be embodied in many different forms and should not be construed as limited to the embodiments set forth herein; rather, these embodiments are provided so that this disclosure may be thorough and complete, and may fully convey the scope of embodiments to those skilled in the art. Among other things, the present invention may be embodied as methods, systems, computer readable media, apparatuses, or devices. Accordingly, the present invention may take the form of an entirely hardware embodiment, an entirely software embodiment, or an embodiment combining software and hardware aspects. The following detailed description may, therefore, not to be taken in a limiting sense.

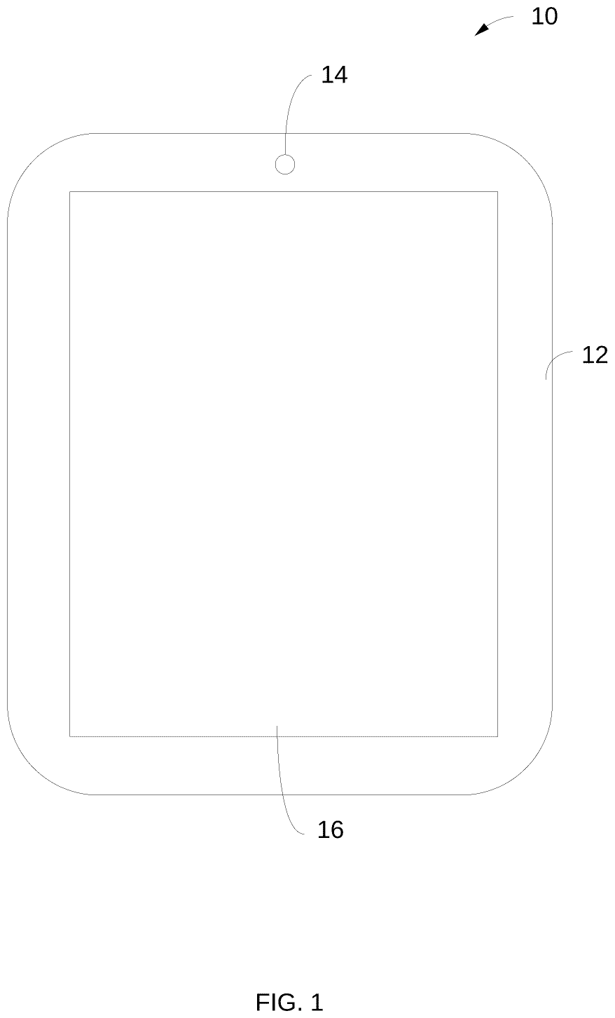

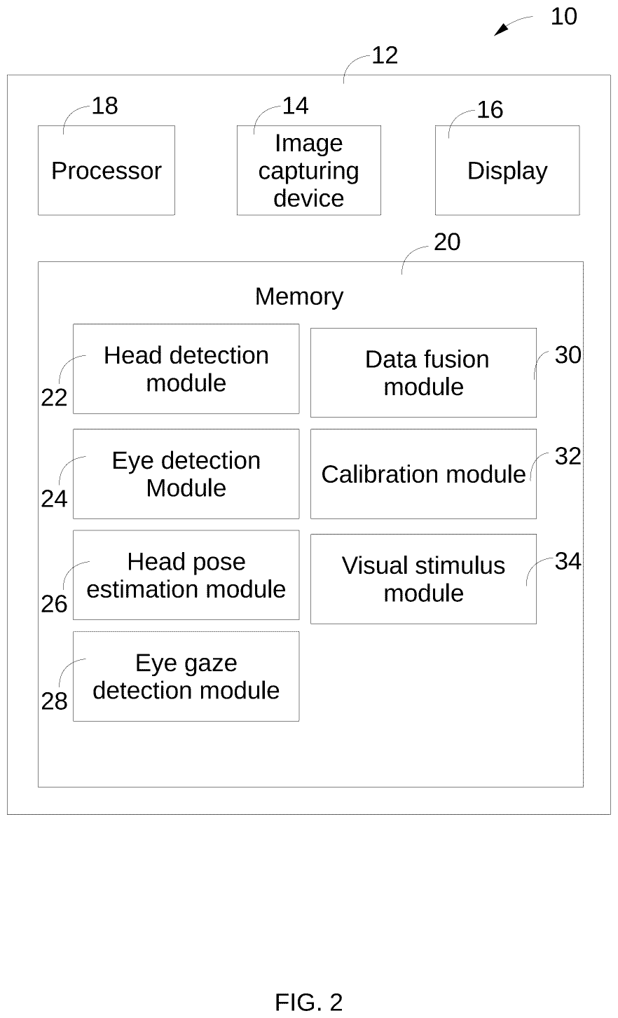

FIG. 1 shows a schematic view of an eye gaze tracking system 10 of the present invention and FIG. 2, shows a block diagram of the system 10. The system 10 comprises a casing 12, an image capturing device 14, a display unit 16, a processor 18, a memory 20. The various components in the system 10 are implemented in hardware, software, or a combination of both hardware and software, including one or more signal processing and/or application-specific integrated circuits.

In certain embodiments, the system is configured to pre-screen, detect or monitor various developmental, cognitive, social, or mental disabilities or abilities of an individual, who is a child aged between 2-6 years old (inclusive).

In certain embodiments, the image capturing device 14, the display unit 16, the processor 18 and the memory 20 are fitted inside the casing 12.

The image capturing device 14 in certain embodiments is an optical sensor equipped with charge-coupled device (CCD) or complementary metal-oxide semiconductor (CMOS) phototransistors. This sensor captures light from the environment through lenses, converting it into image data. It captures still images or video and transmits to the processor 18 and/or the memory 20 for further processing. In various embodiments, the optical sensor may be positioned on the same side as the display unit 16. In some embodiments, the optical sensor can capture image and/or video in at least 2K resolution.

The display unit 16 in certain embodiments is a touch screen that serves as an input and output interface for an operator. It displays visual output, including visual stimulus, graphics, text, icons, and videos, and incorporates virtual buttons and soft keyboards. The touch screen operates through touch-sensitive surfaces or sensors that detect haptic and tactile contact from the user, typically with a finger or stylus. This contact is translated into interactions with displayed user-interface objects, such as soft keys or icons. The touch screen technology can be based on LCD, LPD, or LED technology, although other display technologies may be used. Additionally, the touch screen employs various touch sensing technologies, such as capacitive, resistive, infrared, and surface acoustic wave technologies, to detect contact and movement. These technologies enable the accurate detection of one or more points of contact with the touch screen interface. In other embodiments, the display unit 16 is a screen based on LCD, LPD or LED technology without a touch-sensitive surfaces or sensors, although other display technologies may be used.

Referring to FIG. 2, the processor 18, in one embodiment, runs or executes various software programs and/or sets of instructions stored in the memory 20 to perform various functions for the system and to process data, including the images and videos. In some embodiments, peripherals interface, memory controller, and CPU (central processing unit), GPU (graphic processing unit), TPU (tensor processing unit) or any combination thereof are implemented on a single chip, such as chip. In some other embodiments, they are implemented on separate chips. In yet some embodiments, dynamic caching, hardware-accelerated ray tracing, hardware-accelerated mesh shading, neural engine, machine learning accelerators, or any combination thereof are implemented on the CPU, GPU and/or TPU on the processor 18. In some embodiments, the CPU, GPU and/or TPU contain multiple cores. In some embodiments, the system 10 includes a plurality of processors 18, which includes, but not limited to, CPU, GPU, and TPU.

Memory 20 includes one or more computer-readable storage mediums. The computer-readable storage mediums are, for example, tangible and non-transitory. Memory 20 includes high-speed random access memory (RAM), such as DRAM, SRAM, DDR RAM, and also includes non-volatile memory, such as one or more magnetic disk storage devices, flash memory devices, or other non-volatile solid-state memory devices.

In some examples, a non-transitory computer-readable storage medium of memory 20 is used to store instructions (e.g., for performing aspects of processes described below) for use by or in connection with an instruction execution system, apparatus, or device, such as a computer-based system, processor-containing system, or other system that can fetch the instructions from the instruction execution system, apparatus, or device and execute the instructions. In other examples, the instructions (e.g., for performing aspects of the processes described below) are stored on a non-transitory computer-readable storage medium of a server system or are divided between the non-transitory computer-readable storage medium of memory and the non-transitory computer-readable storage medium of server system.

In some embodiment, the software components stored in memory 20 includes head detection module 22 (or set of instructions), eye detection module 24 (or set of instructions), head pose estimated module 26 (or set of instructions), eye gaze detection module 28 (or set of instructions), data fusion module 30 (or set of instructions), calibration module 32 (or set of instructions) and visual stimulus module 34 (or set of instructions). Furthermore, in some embodiments, the memory 20 stores data, images or videos of the individuals, visual stimulus or combination thereof.

In some embodiments, the execution of the modules involves an execution by the at least one processor 18, which includes, CPU, GPU, TPU or any combination thereof. The processor 18 fetches the instruction module to be executed from memory 20 and proceeds to execute them. The module may be first loaded into the computer's memory, such as RAM, by an operating system prior to the execution by the processor 18.

The head detection module 22 includes various software components to detect the head of the individual in an image and/or video. In some embodiments, the head detection module 22 identifies the head of an individual in an image and/or video using pre-trained machine learning models specifically designed for human head detection tasks. In yet some other embodiments, face landmarks, including, the eyes, nose, mouth and ears of the individual are also identified using the pre-trained machine learning model. The pre-trained machine learning models may include any model that may be configured to identify the head, including but not limited to facial landmarks detection models including, as examples blazeface, mediapipe, opencv haar cascade classifier and dlib.

In some embodiments, the image or video is streamed from the image capturing device 14 and the head detection module 22 detects the head of the individual and/or the facial landmarks in real time.

The eye detection module 24 includes various software components to detect the eye region of the individual in the image or video. In some embodiments, the eye detection module 24 locates at least one eye region by first preprocessing the image or video for better feature extraction. A face detection algorithm then locates the presence and position of a human face. Based on this location and facial geometry knowledge, potential eye regions are identified within the face. Features like intensity, edges, and textures are extracted from these regions to distinguish them from other facial areas. A pre-trained eye classification machine learning model then classifies each candidate region as an eye or not. Based on the detected eye region location, specific features are extracted from the isolated iris region to differentiate it from other eye structures. Specific features may include the shape of the region to identify a near-circular pattern characteristic of the iris, patterns of light and dark regions corresponding to the iris texture, color distribution within the region (as the iris typically exhibits distinct coloration compared to the pupil and sclera) or a combination thereof. A pre-trained iris classification machine learning model then classifies the extracted region as an iris or not.

In some embodiments, the eye detection module 24 further detects whether the eyelid at the detected eye region location is closed (i.e. blinking). The eyelid is determined to be closed when the iris is not detected at the detected eye region location. The total time when the eyelid is closed, the total time when the eyelid is not closed or both is recorded. In certain embodiments, a pre-trained eyelid prediction machine learning model is used to detect whether the eyelid at the detected eye region location is closed. In certain embodiments, the pre-trained eyelid prediction machine learning model was trained on the data associating opened and closed eyelids.

The head pose estimation module 26 includes various software components to estimate the head pose and/or detect if the head pose exceeds a predetermined threshold.

In some embodiments, the head pose estimation module 26 estimates the head pose of the individual by first detecting facial landmarks to identify key points on the face, such as the eyes, nose, mouth and ears of the individual. These landmarks define the facial structure. Second, relevant features are extracted from the detected facial landmarks or the entire face region. For example, geometric features (for instance, distances and angles between the facial landmarks) and/or appearance features (for instance, textures, patterns, or intensity variations with the facial region) are extracted. Third, using a pre-trained machine learning model, including, as examples blazeface, mediapipe, opencv haar cascade classifier and dlib, to estimate the head pose based on the extracted features. The head pose is represented by three rotation angles: (1) yaw: rotation around the vertical axis (turning left/right), (2) pitch: rotation around the horizontal axis (tilting head up/down), and (3) roll: rotation along the line of sight (tilting head sideways).

In some embodiments, the head pose estimation module 26 further compares the estimated head pose with a predetermined threshold. The threshold comprises a first yaw value from about −15 to 15 degrees of yaw and a second pitch value from about −15 to 15 degrees of pitch. If the predicted head pose exceeds the predetermined threshold, the data with respect to the eye gaze direction at a corresponding time point is disregarded/unselected. In other words, if the predicted head pose is within the predetermined threshold, the data with respect to the eye gaze direction at a corresponding time point is selected. In some embodiments, the eye gaze direction detected/predicted is disregarded/unselected. In yet other embodiments, the eye detection module 24, eye gaze detection module 28 or both are not executed once the estimated head pose exceeds the predetermined threshold.

In some embodiments, the threshold comprises a first yaw value from about −15 to 15 degrees of yaw or a second pitch value from about −15 to 15 degrees of pitch.

In some embodiment, the head pose estimation module 26 further records the total time when the head pose is within the predetermined operation head pose threshold range, the total time when the head pose exceeds the predetermined operation head pose threshold range or both.

The eye gaze detection module 28 includes various software components to detect the eye gaze direction of the individual in the image or video and identify the spatial region-of-interest which the individual's eye gaze towards. To detect the eye gaze direction of the individual, in some embodiments, the eye gaze detection module 28 estimates the eye gaze direction based on the location of the identified iris within the detected eye region. The location of the identified iris is determined based on its position relative to the upper, lower eyelids and/or inner corner of the eye.

In other embodiments, a pre-trained eye gaze prediction machine learning model is used to predict gaze direction based on the extracted iris features from the image or video. In some embodiments, the pre-trained eye gaze prediction machine learning model was trained on the data associating iris positions with known gaze directions.

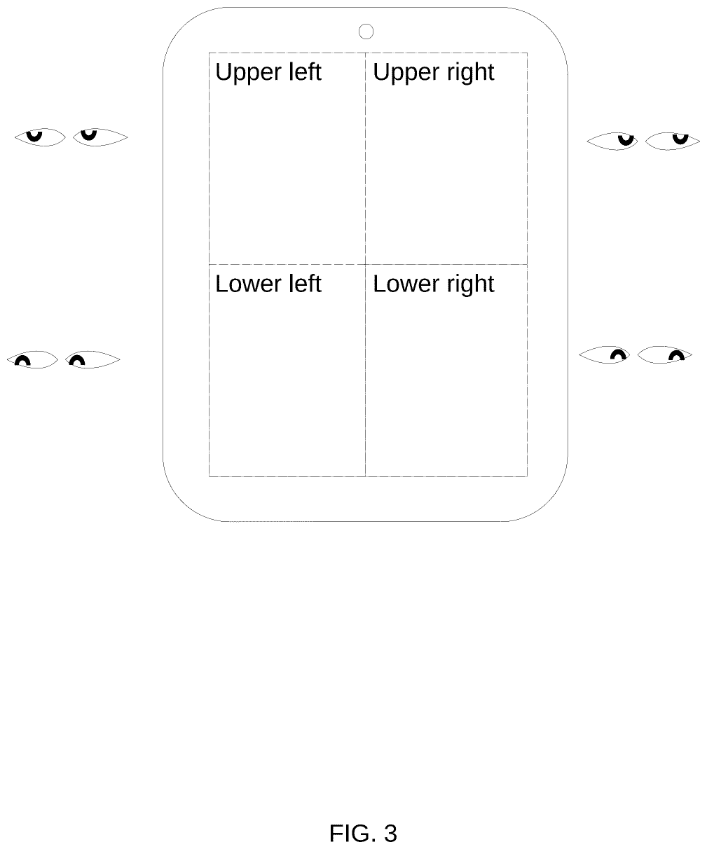

To identify the spatial region-of-interest which the individual's eye gaze towards, the eye gaze direction is mapped to the identified spatial region-of-interests. In some embodiments, the display area of the display unit 16 (alternatively, the visual stimulus) is divided into four quadrants as shown in FIG. 3: the upper left quadrant, the upper right quadrant, the lower left quadrant and the lower right quadrant. Each quadrant is a separated spatial region-of-interest and together form a continuous coverage or substantial continuous coverage over the entire display area or, alternatively, the visual stimulus (i.e. four spatial region-of-interests are defined). For example, lower left eye gaze is mapped to the lower left quadrant (a spatial region-of-interest).

The data fusion module 30 includes various software components to determine an individual's gazing pattern with respect to at least one point-of-interest of the visual stimulus. In particular, including but not limited to, determining one or more parameters with regard to an individual's eye gaze towards at least one point-of-interest of the visual stimulus.

The visual stimulus is a pre-recorded video being displayed on the display unit 16 and the video shows at least one human performer, animal, cartoon character or any combination thereof (collectively performer) performing certain activities. The activity may be telling a favorite story or conducting a cognitive task. The at least one point-of-interest includes, but not limited to, an eye of the performer, a mouth of the performer, interested object, non-interested object in the visual stimulus or a combination thereof. As the visual stimulus is being played on the display unit 16, each point-of-interest falls into one of the quadrants. For example the eyes may fall within the upper right quadrant and the mouth may fall within the lower right quadrant.

A pre-trained visual stimulus classification machine learning model is used to identify frame-by-frame at least one of the point-of-interests from the visual stimulus. The corresponding quadrant (spatial region-of-interest) for each of the point-of-interest for each frame is obtained. The corresponding quadrant data can be obtained real time or frame-by-frame pre-loaded in the memory 20.

The data fusion module 30 then performs a frame-by-frame comparison between the detected gaze direction (or gazed spatial region-of-interest) and the spatial region-of-interests containing the point-of-interests at the same time point. Thereby, the total gazing time or the percentage of total gazing time toward each point-of-interest, or both of them are calculated. The data fusion module 30 may also record the total gazing time or the percentage of total gazing time toward each quadrant (spatial region-of-interest).

In some embodiments, each point-of-interest in the visual stimulus is substantially positioned and assigned to a predetermined quadrant (pre-assigned quadrant) throughout the duration of the visual stimulus. Thereby, the total gazing time or the percentage of total gazing time toward the pre-assigned quadrant corresponding to the assigned point-of-interest is the total gazing time or the percentage of total gazing time toward such point-of-interest. In some embodiments, each point-of-interest is positioned and assigned to an unique quadrant.

Each of the total gazing time or the percentage of total gazing time, or combination thereof may be used as a marker of pre-screening, detecting or monitoring a developmental, cognitive, social, or mental disability or ability in an individual. The data fusion module 30 compares the total gazing time toward the at least one point-of-interest of the video, the percentage of total gazing time toward the at least one point-of-interest of the video, or both against a predetermined threshold. In some embodiments, the threshold includes the norm of the total gazing time and/or the percentage of the total grazing time toward the at least one point-of-interest of the video of a healthy population in the same age group as the individual. In some embodiments, the threshold varies with different age groups. Pre-screening, detecting and/or monitoring results are provided based on the magnitude of the difference and may be generated in real time (i.e. as soon as all the visual stimulus was displayed).

The data fusion module 30 may further generate a report to show the total gazing time toward the at least one point-of-interest of the visual stimulus, the percentage of total gazing time toward the at least one point-of-interest of the visual stimulus, or both. Further, the report may include total gazing time or the percentage of total gazing time toward each spatial region-of-interest. Each of them may be further used as a marker of pre-screening, detecting and/or monitoring a particular developmental, cognitive, social, or mental disability or ability.

In some embodiments, the data fusion module 30 may further calculate the total time and/or percentage of total time for which head pose exceeds the head pose threshold, the total time and/or percentage of total eyelid closed time, combination thereof in respect with the visual stimulus. Each of them may be further used as a marker of pre-screening, detecting or monitoring a developmental, cognitive, social, or mental disability or ability in an individual.

In other embodiments, the data fusion module 30 may further use at least one parameter with regard to an individual's eye gaze towards a spatial region-of-interest, which corresponds to at least one point-of-interest of the visual stimulus to generate gamification elements and/or experiences. The total time and/or percentage of the total time for which head pose exceeds the head pose threshold, the total time and/or percentage of total eyelid closed time, combination thereof in respect with the visual stimulus. Each of them may be further used to generate gamification elements and/or experiences.

In some embodiments, the visual stimulus may include plants and/or graphical objects in various shapes. In some embodiments, visual stimulus is an actual live performance that attracts an individual's visual attention. However, the visual stimulus in the above embodiments are merely illustrative examples, the visual stimulus may include any form of light, image or moving object.

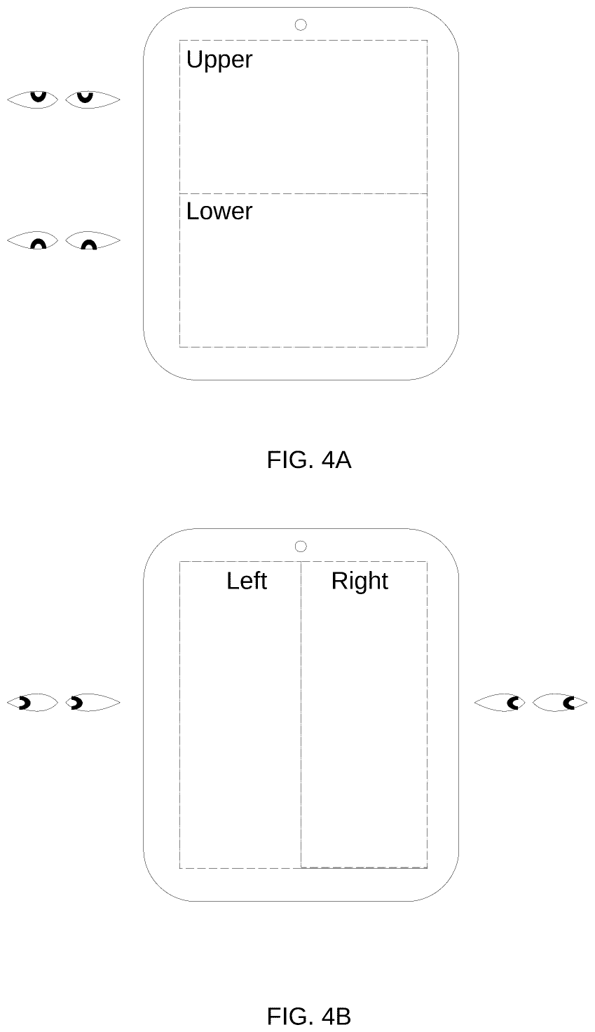

In one embodiment, referring to FIG. 4A, the display area of the display unit 16 (alternatively, the visual stimulus) is divided into upper and lower regions by merging the upper quadrants and lower quadrants respectively. Each region is defined as a spatial region-of-interest. In another embodiment, referring to FIG. 4B, the display unit 16 is divided into left and right regions by merging the left quadrants and the right quadrants respectively. Each region is defined as a spatial region-of-interest. In other embodiments, the display area of the display unit 16 (alternatively, the visual stimulus) is divided into 9 quadrants, including upper left quadrant, upper middle quadrant, upper right quadrant, middle left quadrant, center quadrant, middle right quadrant, lower left quadrant, lower middle quadrant and lower right quadrant. Each quadrant is a separated spatial region-of-interest and together form a continuous coverage or substantial continuous coverage over the entire display area or, alternatively, the visual stimulus (i.e. nine spatial region-of-interests are defined). The above embodiments are merely illustrative examples of arrangements for the spatial region-of-interests. However, the spatial region-of-interest can be arranged in other configurations (for example, different number, different shape, continuous coverage and/or substantial continuous coverage over the entire display area or, alternatively, the visual stimulus) that achieves the same function and falls within the scope of the invention.

The calibration module 32 includes various software components to calibrate the system to the individual before tracking individual eye gaze. The calibration helps the system to consistently identify the iris of the individual for eye gaze tracking. It first utilizes real time facial recognition to detect the face of the individual. It further determines if the distance between the eyes of the individual and the image capturing device 14 is within a first predetermined distance range. In accordance with a determination that the distance not within the first predetermined distance range, prompting to adjust the distance. It also determines if a head pose of the individual is within a predetermined orientation range and in accordance with a determination that the head orientation is not within the predetermined range, prompting to adjust the head orientation. The calibration module 32 is also configured to cause the display unit 16 to display desired colours to catch the individual's and/or operator's attention for calibration.

In some embodiments, the first predetermined distance range is about 25-35 cm from the image capturing device 14. In other embodiments, the first predetermined distance is about 30-40 cm from the image capturing device 14. In yet some embodiments, the predetermined orientation range includes a first yaw range of about −15 to 15 degrees of yaw, a first pitch range of about −15 to 15 degrees of pitch or both.

The visual stimulus module 34 includes various software components to cause the display unit 16 to display the visual stimulus. In some embodiments, the visual stimulus is pre-loaded into the memory 20. The visual stimulus module is configured to load the visual stimulus and display it on the display unit 16. In yet another embodiment, the visual stimulus module 34 steams the visual stimulus from the network and shows it on the display unit 16. In some embodiments, each point-of-interest in the visual stimulus is assigned and positioned and assigned in an unique spatial region-of-interest.

In some embodiments, all the modules may be executed in real time as the image or video is streamed from the image capturing device 14. It includes, but not limited to, the head detection, the head pose estimation, the eye region detection, the eyelid detection, the eye gaze direction and/or spatial region-of-interest identification, and the determination of one or more parameters with regard to an individual's eye gaze towards at least one point-of-interest of the visual stimulus. In other embodiments, all the modules besides the calibration module 32 and visual stimulus module 34 may be run by the processor 18 after the image or video was fully captured by the image capturing device 14 and stored in the memory 20. In some embodiments, the processor 18, the memory 20 or both are located in a remote server system (e.g. cloud server).

In some embodiments, the system 10 is in the form of a tablet computer. In some embodiments, the screen size is preferably in a range of ranging from 9.7 to 12.9 inch diagonally. It is noted that other screen sizes may be used. In yet another embodiment, the image capturing device 14, the display unit 16, the processor 18, and the memory 20 are physically separated from each other and connected via a digital communication data network. For example, the processor 18, the memory 20 or both may be located at a remote server system (e.g. cloud server).

In some embodiments, the network may include wire communication or wireless communication. In some embodiments, wire networks may include local area network (LAN), an Internet or a combination thereof. In some embodiments, the wireless communication may include the WI-FI network, a cellular network, an Internet or a combination thereof. In a further embodiment, wireless communication may include satellite communication. In yet another example, wireless communication may include BLUETOOTH, NFC, or other short-range wireless communication protocols. In yet another example, the digital communication data network may be any wireless and/or wired communication protocols.

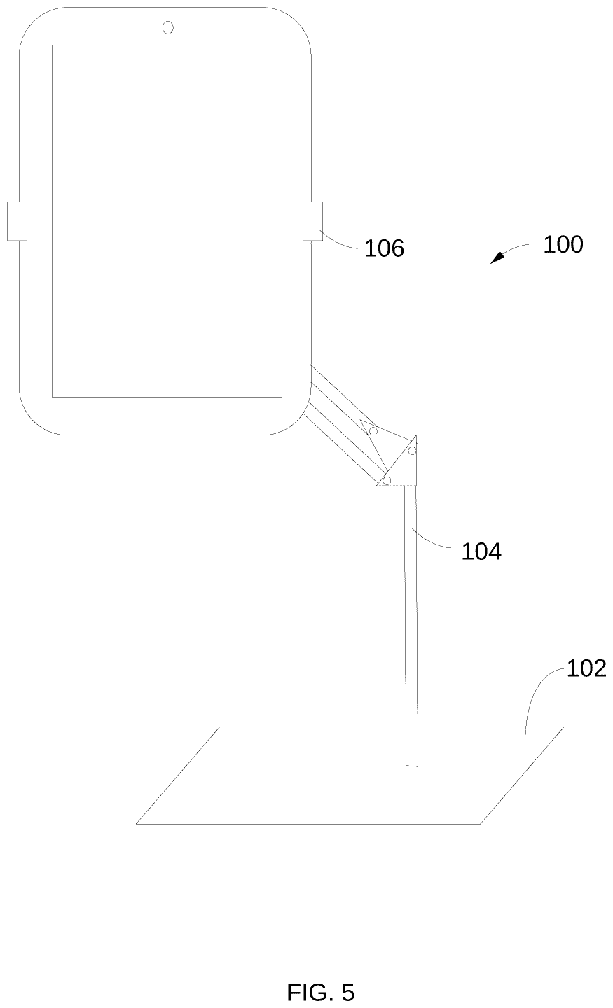

FIG. 5 shows a schematic view of an eye gaze tracking system 100 including a floor stand holder according to certain embodiments of the present invention. The casing 12, image capturing device 14, display unit 16, processor 18, and memory 20 of this certain embodiment is described above. The floor stand holder comprises base 102, an adjustable arm 104 extending from the base and a holding mechanism 106 to fix the casing 12, the display unit 16 and the image capturing device 14 at a predetermined height above the ground. In some embodiments, the predetermined height is about the eye level of the individual. In other embodiments, the predetermined height is defined as the height where the image capturing device 14 is about 0-10 cm above the individual's eyes.

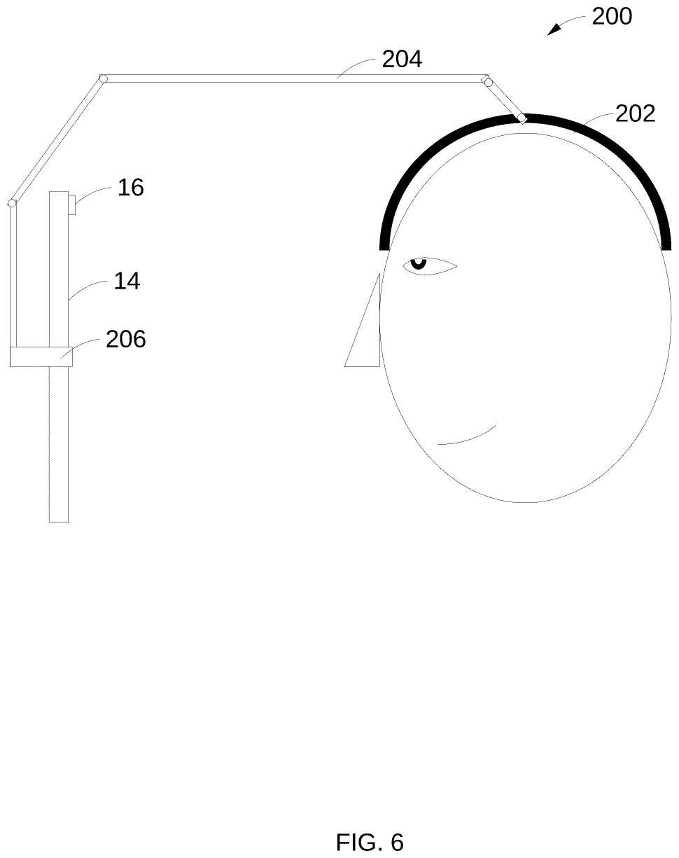

FIG. 6 shows a schematic view of an eye gaze tracking system 200 including a holder according to one embodiment of the present invention. The casing 12, image capturing device 14, display unit 16, processor 18, and memory 20 of this certain embodiment is described above. The holder comprises a cap 202 configured to fit the individual head. The holder further comprises an adjustable arm 204 connected to the cap at one end and extended away from the front of the cap. A holding mechanism 206 is attached to the other end of the adjustable arm and is configured to fix the casing 12, the display unit 14 and the image capturing device 14 at a predetermined position above the ground. In some embodiments, the predetermined position is about the eyes level of the individual and the image capturing device 14 is about 25-35 cm from the eyes of the individual. In other embodiments, the predetermined position is about the eyes level of the individual and the image capturing device 14 is about 30-40 cm from the eyes of the individual. In other embodiments, the image capturing device 14 is about 0-10 cm above the individual's eyes.

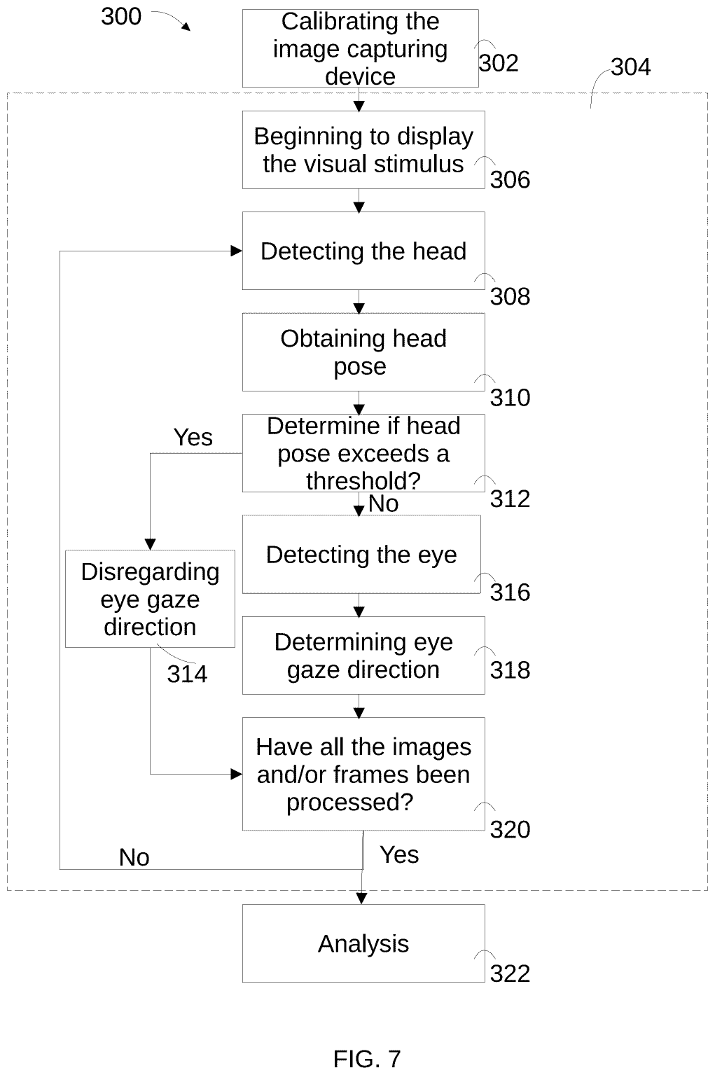

FIG. 7 shows a flowchart with computer-implemented functions for identifying eyes gaze of an individual corresponding to at least one point-of-interest in a visual stimulus provided to the individual according to certain embodiments.

Process 300 begins at calibration step 302, where the system 10 is calibrated to the individual. In this step, the distance between the image capturing device 14 and the individual is determined. If it is not within a predetermined distance range, prompting the individual and/or the operator to adjust the distance between the individual and the image capturing device 14 such that they are separated within the predetermined distance range. Further, the head pose of the individual is determined. If it is not within the predetermined orientation, prompting the individual and/or operator to adjust the head orientation of the individual.

The operator can be a caregiver who helps the individual to participate in the process. If the individual is a child, the caregiver can let the individual sit on his/her lap and help the individual to adjust the position and head pose to meet the requirements during the calibration step.

In some embodiments, the distance is determined based on a live image and/or video of the individual. In these embodiments, the head of the individual is identified employing real time facial recognition techniques. The distance between the head of the individual and the image capturing device 14 is estimated by comparing the head size within the image and/or video against a predetermined dimension. Comparing the estimated distance with the predetermined distance range to determine if the head of the individual is within the acceptable distance range. Providing feedback to the individual and/or operator to indicate whether the head of the individual is within the predetermined distance range, such as through visual indicators or notifications. In some embodiments, the predetermined distance is about 25-35 cm from the image capturing device 14 at the individual's eyes level. In other embodiments, the predetermined distance is about the eyes level of the individual and the image capturing device 14 is about 30-40 cm from the eyes of the individual.

In some embodiments, the head orientation is determined based on an image and/or video of the individual. In these embodiments, the head of the individual is identified employing real time facial recognition techniques. Then, the face landmarks on the detected head, such as the eyes, nose, and mouth are identified using facial landmark detection techniques. The orientation of the head is estimated based on the positions of the detected facial landmarks. Comparing the estimated orientation of the head of the individual with a predetermined orientation range. Providing feedback to the individual and/or operator to indicate whether the head of the individual is within the predetermined orientation, such as through visual indicators or notifications. In yet some embodiments, the predetermined orientation range include a first yaw value about −15 to 15 degrees of yaw, a second pitch value about −15 to 15 degrees of pitch or a combination thereof.

The process 300 then begins to collect response data 304 of the individual after the calibration step 302. Collecting response data involves multiple steps, which begins with visual stimulus step 306. At this step 306, a visual stimulus is shown to the individual. In some embodiments, the visual stimulus is a video and is pre-loaded into the memory 20. The visual stimulus is loaded from the memory 20 when it is shown to the individual on the display unit 16. In yet other embodiments, the visual stimulus is a video and is streamed from the network when it is shown to the individual on the display unit 16. The video may include at least one human performer, animal, cartoon character or any combination thereof (collectively performer) performing certain activities. The activity may be telling a story or conducting a cognitive task.

In some embodiments, the visual stimulus includes plants and/or graphical objects in various shapes. In some embodiments, visual stimulus is an actual live performance that attracts an individual's visual attention. However, the visual stimulus in the above embodiments are merely illustrative examples, the visual stimulus may include any form of light, image or moving object.

In some embodiments, the head and the head pose of the individual are determined based on an image and/or video of the individual frame-by-frame. At detecting head step 308, the head of the individual is identified employing real time facial recognition techniques. In some embodiments, face landmarks, including, the eyes, nose, mouth and ears of the individual are also identified using the pre-trained machine learning model. The pre-trained machine learning models may include any model that may be configured to identify the head, including but not limited to face landmarks detection models including, as examples blazeface, mediapipe, opencv haar cascade classifier and dlib.

Then at obtaining head pose step 310, the face landmarks including, the eyes, nose, mouth and ears of the individual are identified using facial landmark detection techniques. These landmarks define the facial structure. For example, geometric features (for instance, distances and angles between the facial landmarks) and/or appearance features (for instance, textures, patterns, or intensity variations with the facial region) are extracted. The orientation of the head is estimated based on the positions of the detected facial landmarks, using a pre-trained machine learning model including, as examples blazeface, mediapipe, opencv haar cascade classifier and dlib, to estimate the head pose based on the extracted features. The head pose is represented by three rotation angles: (1) yaw: rotation around the vertical axis (turning left/right), (2) pitch: rotation around the horizontal axis (tilting head up/down), and (3) roll: rotation along the line of sight (tilting head sideways).

At determining head pose step 312, the estimated head pose of the individual is compared with a predetermined operation head pose threshold range. The threshold includes a first yaw value of about −15 to 15 degrees of yaw, a second pitch value of about −15 to 15 degrees of pitch or both. If the estimated head pose does not exceed the threshold, the process continues at the eye detection step 316, where at least one eye will be detected. If the estimated head pose exceeds the threshold, the process disregards/unselects the data with respect to the eye gaze direction at a corresponding time point at the disregarding step 314. At this step, the data with respect to the gaze direction is disregarded/unselected by skipping the eye detection step 316 and eye gaze detection step 318 for that frame (i.e. once the estimated head pose exceeds the predetermined threshold).

In some embodiment, the determining head pose step 312 further records the total time when the head pose is within the predetermined operation head pose threshold range, the total time when the head pose exceeds the predetermined operation head pose threshold range or both.

At eye detection step 316, at least one eye region is located by first preprocessing the image or video for better feature extraction. A face detection algorithm then locates the presence and position of a human face. Based on this location and facial geometry knowledge, potential eye regions are identified within the face. Features like intensity, edges, and textures are extracted from these regions to distinguish them from other facial areas. A pre-trained eye classification machine learning model then classifies each candidate region as an eye or not. Based on the detected eye region location, specific features are extracted from the isolated iris region to differentiate it from other eye structures. Specific features may include the shape of the region to identify a near-circular pattern characteristic of the iris, patterns of light and dark regions corresponding to the iris texture, color distribution within the region (as the iris typically exhibits distinct coloration compared to the pupil and sclera) or a combination thereof. A pre-trained iris classification machine learning model then classifies the extracted region as an iris or not.

In some embodiments, the eye detection step 316 further detects whether the eyelid at the detected eye region location is closed (i.e. blinking). The eyelid is determined to be closed when the iris is not detected at the detected eye region location. The total time when the eyelid is closed, the total time when the eyelid is not closed or both is recorded. In certain embodiments, a pre-trained eyelid prediction machine learning model is used to detect whether the eyelid at the detected eye region location is closed. In certain embodiments, the pre-trained eyelid prediction machine learning model was trained on the data associating opened and closed eyelids.

At eye gaze detection step 318, the eye gaze direction is estimated based on the location of the identified iris within the detected eye region. The location of the identified iris is determined based on its position relative to the upper, lower eyelids and/or inner corner of the eye. In some embodiments, a pre-trained eye gaze prediction machine learning model is used to predict gaze direction based on the extracted iris features from the image or video. In some embodiments, the pre-trained eye gaze prediction machine learning model was trained on the data associating iris positions with known gaze directions.

In addition, the spatial region-of-interest which the individual's eye gaze towards is also identified at eye gaze detection step 318. At this step, the eye gaze direction is mapped to the identified spatial region-of-interests. In some embodiments, the display area of the display unit 16 (alternatively, the visual stimulus) is divided into four quadrants as shown in FIG. 3: the upper left quadrant, the upper right quadrant, the lower left quadrant and the lower right quadrant. Each quadrant is a separated spatial region-of-interest and together form a continuous coverage or substantial continuous coverage over the entire display area or, alternatively, the visual stimulus (i.e. four spatial region-of-interests are defined). For example, lower left eye gaze is mapped to the lower left quadrant (a spatial region-of-interest).

At data processing verification step 320, it determines whether all the images and/or frames of the video are processed. If not all the images and/or frames of the video are processed, steps 306-318 (i.e. the detecting head step, obtaining head pose step, determining head pose step, eye detection step and eye gaze detection step) are repeated for the next consecutive image and/or frame until all the images and/or frames of the video are processed. If all the images and/or frames of the video are processed, the collection of response data from the individual is completed. The process then continues to response data analysis process 322.

In some other embodiments, the disregarding step 314 does not skip the eye detection step 316 and eye gaze detection step 318. Instead, the eye detection step 316 and eye gaze detection step 318 are executed. The disregarding step 314 is executed after the eye detection step 316 and eye gaze detection step 318 are executed. The predicted gaze direction and spatial region-of-interest are disregarded/unselected by the disregarding step 314.

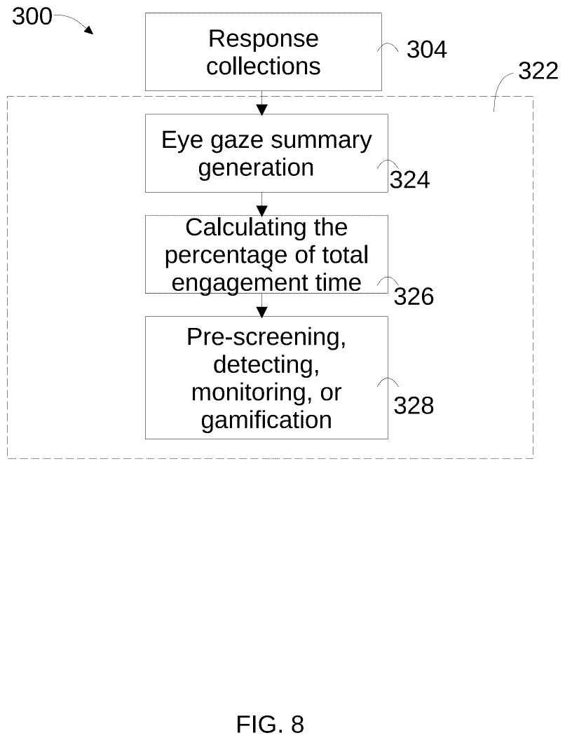

Referring to FIG. 8, response data analysis process 322 is shown according to certain embodiments of the present invention. The analysis begins with eye gaze summary generation step 324. At the eye gaze summary generation step 324, one or more parameters with regard to an individual's eye gaze towards a spatial region-of-interest corresponding to at least one point-of-interest of the visual stimulus is determined.

The visual stimulus is a video being displayed on the display unit 16 and the video shows a performer performing certain activities. The at least one point-of-interest includes, but not limited to, an eye of the performer, a mouth of the performer, interested object, non-interested object in the visual stimulus or a combination thereof. As the visual stimulus is being played on the display unit, each point-of-interest falls into one of the quadrants. For example the eyes may fall within the upper right quadrant and the mouth may fall within the lower right quadrant.

A pre-trained visual stimulus classification machine learning model is used to identify frame-by-frame at least one of the points-of-interest from the visual stimulus. The corresponding quadrant (spatial region-of-interest) for each of the point-of-interest for each frame is obtained. The corresponding quadrant data can be obtained real time or frame-by-frame pre-loaded in the memory 20.

A frame-by-frame comparison between the detected gaze direction (or gazed spatial region-of-interest) and the spatial region-of-interests containing the point-of-interests at a same time point is performed. The total gazing time toward each point-of-interest is thereby obtained.

At the parameter calculation step 326, the percentage of total gazing time toward each point-of-interest is calculated. The total gazing time or the percentage of total gazing time toward each quadrant (spatial region-of-interest) is also recorded. In some embodiments, each point-of-interest in the visual stimulus is substantially positioned and assigned to a predetermined quadrant (pre-assigned quadrant) throughout the duration of the visual stimulus. Thereby, the total gazing time or the percentage of total gazing time toward the pre-assigned quadrant corresponding to the assigned point-of-interest is the total gazing time or the percentage of total gazing time toward such point-of-interest. In some embodiments, each point-of-interest is positioned and assigned to an unique quadrant.

In some embodiments, the parameter calculation step 326 further calculate the total time and/or percentage of the total time for which the head pose exceeds the operation head pose threshold, the total time and/or percentage of total time for which the eyelid is closed, combination thereof in respect with the visual stimulus.

At the result generating step 328, the total gazing time toward the at least one point-of-interest of the video, the percentage of total gazing time toward the at least one point-of-interest of the video, or both are compared against a predetermined threshold. In some embodiments, the threshold includes the norm of the total gazing time and/or the percentage of total gazing time toward the at least one point-of-interest of the video of a healthy population in the same age group as the individual. In some embodiments, the threshold varies with different age groups. Pre-screening, detecting and/or monitoring results are provided based on the magnitude of the difference and may be generated in real time (i.e. as soon as all the visual stimulus was displayed).

Further, in certain embodiments, the total time and/or percentage of total time for which the head pose exceeds the operation head pose threshold, the total time and/or percentage of total time for which eyelid is closed, or combination thereof are compared against their respective predetermined threshold. Similarly, the threshold includes the norm of the total time for which head pose exceeds the operation head pose threshold and closed eyelids of a healthy population in the same age group as the individual. Pre-screening, detecting and/or monitoring results are provided based on the magnitude of the difference and may be generated in real time (i.e. as soon as all the visual stimulus was displayed).

For example, if the visual stimulus shows a human performer working on puzzles at the right hand side of the display area of the display unit and the percentage of total gazing time towards quadrants on the left hand side is higher than the norm of a healthy population, it indicates that the individual may suffer from autistic. Similarly, if the percentage of total time for which the head pose and/or the percentage of total eyelid closed time are higher than a norm of a healthy population in the age group of the individual, the individual may suffer from disorder.

At this step 328, a report may be also generated to show the total gazing time toward the at least one point-of-interest of the video, the percentage of total gazing time toward the at least one point-of-interest of the video, or both. Further, the report may include total gazing time or the percentage of total gazing time toward each spatial region-of-interest. In addition, the report may include pre-screening, detecting and/or monitoring results for particular developmental, cognitive, social, or mental disability or ability. In some embodiments, the total time and/or percentage of total time for which the head pose exceeds and/or within the operation head pose threshold, the total time and/or percentage of total time for which the eyelid is closed, or combination thereof may also be included in the report.

In other embodiments, the at least one parameter with regard to an individual's eye gaze towards a spatial region-of-interest, which corresponds to at least one point-of-interest of the visual stimulus can be used to generate gamification elements and/or experiences.

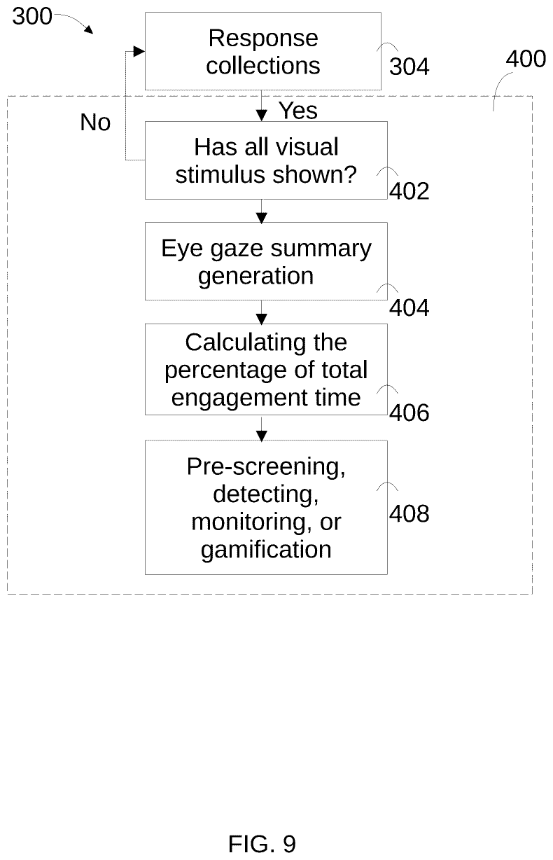

Referring to FIG. 9, response data analysis process 400 is shown in accordance with other embodiments of the present invention. In these embodiments, the eye gaze data analysis utilizes a plurality set of individuals's eyes gaze direction data, wherein each set corresponds to an independently visual stimulus. The eye gaze data analysis process 400 begins with visual stimulus verification step 402, which determines if all the visual stimuli were shown to the individual. If not all the visual stimuli were shown to the individual, the process performs to collect eye gaze directions data 304 for another visual stimulus. If all the visual stimuli were shown to the individual, the process follows with eye gaze summary generation step 404.

At the eye gaze summary generation step 404, one or more parameters with regard to an individual's eye gaze towards a spatial region-of-interest corresponding to at least one point-of-interest of each visual stimulus is determined. For each set of visual stimuli, a frame-by-frame comparison between the detected gaze direction (or gazed spatial region-of-interest) and the spatial region-of-interests containing the point-of-interests at a same time point is performed. For each set of visual stimuli, the total gazing time toward each point-of-interest is thereby obtained.

In some embodiments, two or more quadrants can be merged to form a region. For example, the upper left quadrant and the upper right quadrant can be merged to form an upper region and the lower left quadrant and the lower right quadrant can be merged to form a lower region. Similarly, the upper left quadrant and the lower left quadrant can be merged to form a left region and the upper right quadrant and the lower right quadrant can be merged to form a right region. At the eye gaze summary generation step, the total gazing time toward the upper region and the lower region for the first visual stimulus can be obtained. Similarly, the total gazing time toward the left region and the right region for the second visual stimulus can be obtained.

At the parameter calculation step 406, for each set of visual stimuli, the percentage of total gazing time toward each point-of-interest is calculated. The total gazing time or the percentage of total gazing time toward each quadrant (spatial region-of-interest) is also recorded. In some embodiments, the point-of-interests in the visual stimulus are substantially positioned and assigned to a predetermined quadrant (pre-assigned). Thereby, the total gazing time or the percentage of total gazing time toward the pre-assigned quadrant corresponding to the assigned point-of-interest is the total gazing time or the percentage of total gazing time toward such point-of-interest. Similarly, if two quadrant is merged to form a region (e.g. left region, right region, upper region and lower region), the total gazing time or the percentage of total gazing time toward the quadrants under their respective region are grouped and summed into the total gazing time or the percentage of total gazing time toward the region. The total gazing time or the percentage of total gazing time toward the region containing a point-of-interest is the total gazing time or the percentage of total gazing time toward such point-of-interest.

In some embodiments, the parameter calculation step 406 further calculates the total time and/or percentage of total head pose time, the total time and/or percentage of total eyelid closed time, or both for each visual stimulus.

At the result generating step 408, for each set of visual stimulus, the total gazing time toward the at least one point-of-interest of the video, the percentage of total gazing time toward the at least one point-of-interest of the video, or both are compared against a predetermined threshold. In some embodiments, the threshold includes the norm of the total gazing time and/or percentage of total gazing time toward the at least one point-of-interest of the video of a healthy population in the same age group as the individual. Similar to step 328, in certain embodiments, for each set of visual stimuli, the total time and/or percentage of total time for which head pose within/exceeds the head pose threshold, the total time and/or percentage of total eyelid closed time, or combination thereof are compared against their respective predetermined threshold.

The threshold for each visual stimulus may be different. The threshold for different age groups may vary. The comparison results can be combined to provide pre-screening, detecting and/or monitoring results in real time.

The result generating step 408 may further generate a score for the result for each visual stimulus and generate a combined score. If the combined score may be compared against a predetermined combined score threshold.

At this step 408, a report may be also generated to show the total gazing time toward the at least one point-of-interest of the video, the percentage of total gazing time toward the at least one point-of-interest of the video, or both for each visual stimulus set. Alternatively, the report may include total gazing time or the percentage of total gazing time toward the spatial region-of-interest for each visual stimulus set.

The at least one parameter with regard to an individual's eye gaze towards a spatial region-of-interest, which corresponds to at least one point-of-interest of the visual stimulus can be used to generate gamification elements and/or experiences. For example, it may provide matching and mapping experience to improve the interaction experience through eye gaze, e.g. determining if the food corresponding to an animal appearing on the visual stimulus is correctly selected through eye gaze.

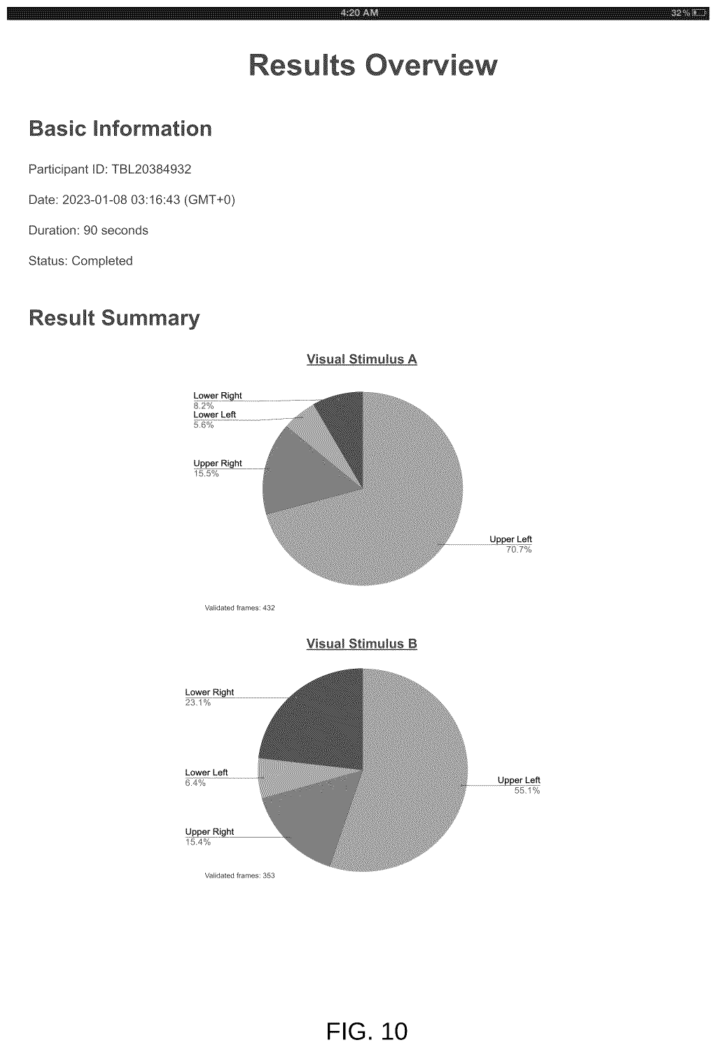

FIG. 10 illustrates an exemplary report. The report includes the percentage of total gazing time toward each of the four quadrants for each visual stimulus. For the first visual stimulus, a point-of-interest is strategically positioned and assigned on only one region of the display area/visual stimulus of the display unit throughout the duration of the first visual stimulus, wherein the display area/visual stimulus is divided into two regions along the vertical axis. For example, the point-of-interest is a performer working on a certain activity. The first run helps identify the duration of the individual looking at the point-of-interest along the horizontal axis.

Similarly, for the second visual stimulus, a point-of-interest is strategically positioned and assigned on the one region of the display area of the display unit/visual stimulus throughout the duration of the visual stimulus, wherein the display area/visual stimulus is divided into two regions along the horizontal axis. For example, the point-of-interest is the eyes of a performer. The second run helps identify the duration of the individual looking at the point-of-interest along the vertical axis.

As used herein, the term “about” means a range around a given value wherein the resulting value is the same or substantially the same (e.g., within 10%, 5% or 1%) as the expressly recited value. In one embodiment, “about” means within 10% of a given value or range. In another embodiment, the term “about” means within 5% of a given value or range. In another embodiment, the term “about” means within 1% of a given value or range.

The example embodiments may include additional devices and networks beyond those shown. Further, the functionality described as being performed by one device may be distributed and performed by two or more devices. Multiple devices may also be combined into a single device, which may perform the functionality of the combined devices.

The various participants and elements described herein may operate one or more computer apparatuses to facilitate the functions described herein. Any of the elements in the above-described Figures, including any servers, user devices, or databases, may use any suitable number of subsystems to facilitate the functions described herein.

Any of the software components or functions described in this application, may be implemented as software code or computer readable instructions that may be executed by at least one processor using any suitable computer language such as, for example, Java, C++, or Python using, for example, conventional or object-oriented techniques.

The software code may be stored as a series of instructions or commands on a non-transitory computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus and may be present on or within different computational apparatuses within a system or network.

It may be understood that the present invention as described above may be implemented in the form of control logic using computer software in a modular or integrated manner. Based on the disclosure and teachings provided herein, a person of ordinary skill in the art may know and appreciate other ways and/or methods to implement the present invention using hardware, software, or a combination of hardware and software.

One or more features from any embodiment may be combined with one or more features of any other embodiment without departing from the scope embodiments. A recitation of “a”, “an” or “the” is intended to mean “one or more” unless specifically indicated to the contrary. Recitation of “and/or” is intended to represent the most inclusive sense of the term unless specifically indicated to the contrary.

One or more of the elements of the present system may be claimed as means for accomplishing a particular function. Where such means-plus-function elements are used to describe certain elements of a claimed system it may be understood by those of ordinary skill in the art having the present specification, figures and claims before them, that the corresponding structure includes a computer, processor, or microprocessor (as the case may be) programmed to perform the particularly recited function using functionality found in a computer after special programming and/or by implementing one or more algorithms to achieve the recited functionality as recited in the claims or steps described above. As would be understood by those of ordinary skill in the art that algorithm may be expressed within this disclosure as a mathematical formula, a flow chart, a narrative, and/or in any other manner that provides sufficient structure for those of ordinary skill in the art to implement the recited process and its equivalents.

While the present disclosure may be embodied in many different forms, the drawings and discussion are presented with the understanding that the present disclosure is an exemplification of the principles of one or more inventions and is not intended to limit any one embodiment to the embodiments illustrated.

The disclosure, in its broader aspects, is therefore not limited to the specific details, representative system and methods, and illustrative examples shown and described above. Various modifications and variations may be made to the above specification without departing from the scope or spirit of the present disclosure, and it is intended that the present disclosure covers all such modifications and variations provided they come within the scope of the following claims and their equivalents.

What is claimed is:

1. A method for identifying an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element.

2. The method of claim 1, further comprising the steps of
defining a plurality of spatial region-of-interests in the visual stimulus wherein the spatial region-of-interests form a substantial continuous coverage over the entire visual stimulus,
wherein an individual's eye gaze towards at least one point-of-interest in the visual stimulus is obtained by determining the individual's eye gaze towards a pre-assigned spatial region-of-interest corresponding to the respective point-of-interest, and
wherein the one or more eye gaze parameters comprise a total gazing time for which the individual's eye gaze is towards the pre-assigned spatial region-of-interest.

3. The method of claim 2 further comprising the step of:
obtaining one or more head pose parameters with regard to individual's head pose based on the head pose comparison step,
wherein the one or more head pose parameters comprise a total time for which the individual's head pose exceeds the threshold, and
wherein the one or more head pose parameters are (a) a marker of pre-screening, detecting or monitoring a developmental, cognitive, social, or mental disability or ability or (b) used to generate a gamification element.

4. The method of claim 3 further comprising the step of
calculating the percentage of total gazing time corresponding to at least one of the spatial region-of-interest,
calculating the percentage of total time for which the individual's head pose exceeds the threshold,
wherein each of the percentage of total gazing time and the percentage of total time that the head pose exceeds the threshold are (a) a marker of pre-screening, detecting or monitoring a developmental, cognitive, social, or mental disability or ability or (b) used to generate a gamification element.

5. The method of claim 4, wherein each unique spatial region-of-interest represents a quadrant of the visual stimulus.

6. The method of claim 5, wherein the head pose threshold range is about −15 to 15 degrees of yaw, about −15 to 15 degrees of pitch or a combination thereof.

7. The method of claim 6, wherein the point-of-interest is selected from a group consisting of an eye in the visual stimulus, a mouth in the visual stimulus, interested object in the visual stimulus, non-interested object in the visual stimulus and a combination thereof.

8. The method of claim 7 further comprises the steps of
providing a display unit and an image capturing device in front of the individual such that the individual is eye facing the display and the image capturing device;
calibrating the image capturing device to the individual, wherein the calibration step further comprises the steps of
determining if the distance between the individual and the image capturing device is within first predetermined distance range;
in accordance with a determination that the distance not within the predetermined distance range, prompting to adjust the distance;
determining if a head orientation of the individual is within a predetermined orientation range;
in accordance with a determination that the head orientation not within the predetermined orientation range, prompting to adjust the head orientation; and
displaying the visual stimulus on the display unit after the calibration step.

9. A non-transitory computer-readable storage medium storing one or more programs, the one or more programs comprising instructions, which when executed by one or more processors of an electronic device, cause the electronic device to identify an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element.

10. The non-transitory computer-readable storage medium of claim 9, wherein the programs further comprise instructions, which when executed by one or more processors of the electronic device, cause the electronic device to:
defining a plurality of spatial region-of-interests in the visual stimulus wherein the spatial region-of-interests from a substantial continuous coverage over the entire visual stimulus;
wherein individual's eye gaze towards at least one point-of-interest in the visual stimulus is obtained by determining individual's eye gaze towards a pre-assigned spatial region-of-interest corresponding to the respective point-of-interest, and
wherein the one or more eye gaze parameters comprise a total gazing time for which the individual's eye gaze is towards the pre-assigned spatial region-of-interest.

11. The non-transitory computer-readable storage medium of claim 10, wherein the programs further comprise instructions, which when executed by one or more processors of the electronic device, cause the electronic device to:
obtaining one or more head pose parameters with regard to individual's head pose based on the head pose comparison step,
wherein the one or more head pose parameters comprise a total time for which the individual's head pose exceeds the threshold, and
wherein the one or more head pose parameters are (a) a marker of pre-screening, detecting or monitoring a developmental, cognitive, social, or mental disability or ability or (b) used to generate a gamification element.

12. The non-transitory computer-readable storage medium of claim 11, wherein the programs further comprise instructions, which when executed by one or more processors of the electronic device, cause the electronic device to:
calculating the percentage of total gazing time corresponding to at least one of the spatial region-of-interest,
calculating the percentage of total time for which the individual's head pose exceeds the threshold,
wherein each percentage of total gazing time and the percentage of total time that the head pose exceeds the threshold are (a) a marker of pre-screening, detecting or monitoring a developmental, cognitive, social, or mental disability or ability or (b) used to generate a gamification element.

13. The non-transitory computer-readable storage medium of claim 12,
wherein each unique spatial region-of-interest represents a quadrant of the visual stimulus, the head pose threshold range is about −15 to 15 degrees of yaw, about −15 to 15 degrees of pitch or a combination thereof, and
wherein the point-of-interest is selected from a group consisting of an eye in the visual stimulus, a mouth in the visual stimulus, interested object in the visual stimulus, non-interested object in the visual stimulus and a combination thereof.

14. The non-transitory computer-readable storage medium of claim 13, wherein the programs further comprise instructions, which when executed by one or more processors of the electronic device, cause the electronic device to:
calibrate an image capturing device to the individual, wherein the calibration step further comprises the steps of
determine if the distance between the individual and the image capturing device is within first predetermined distance range;
in accordance with a determination that the distance not within the predetermined distance range, prompting to adjust the distance;
determine if a head orientation of the individual is within a predetermined orientation range;
in accordance with a determination that the head orientation not within the predetermined orientation range, prompting to adjust the head orientation; and
display the visual stimulus on a display unit after the calibration step.

15. An eye gaze tracking system for identifying an individual's gaze pattern in response to a visual stimulus comprising:
at least one image capturing device to capture a head and at least one eye of the individual;
at least one display unit to display the visual stimulus to the individual;
at least one processor to receive and process the captured images and/or video from the image capturing device;
a head detection module to detect a head of the individual from the captured images and/or video;
an eye detection module to detect at least one eye region of the individual from the captured images and/or video;
an eye gaze detection module to determine an eye gaze direction of the individual from the detected eye region;
a head pose estimation module to obtain an head pose and compare the obtained head pose with a predetermined threshold range and if the obtained head pose falls within the threshold range, selecting an eye gaze direction of the individual associated with such head pose; and
a data fusion module to obtaining one or more eye gaze parameters with regard to individual's eye gaze towards at least one point-of-interest of the visual stimulus using the selected eye gaze direction based on the head pose comparison step,
wherein the one or more eye gaze parameters is (a) a marker of pre-screening, detecting or monitoring a developmental, cognitive, social, or mental disability or ability or (b) used to generate a gamification element.

16. An eye gaze tracking system of claim 15 further comprising a non-transitory computer-readable storage medium storing one or more programs, the one or more programs comprising instructions, which when executed by one or more processors of an electronic device, cause the electronic device to identify an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element; and
wherein the programs further comprise instructions, which when executed by one or more processors of the electronic device, cause the electronic device to:
defining a plurality of spatial region-of-interests in the visual stimulus wherein the spatial region-of-interests from a substantial continuous coverage over the entire visual stimulus;
wherein individuals eye gaze towards at least one point-of-interest in the visual stimulus is obtained by determining individual's eye gaze towards a pre-assigned spatial region-of-interest corresponding to the respective point-of-interest, and
wherein the one or more eye gaze parameters comprise a total gazing time for which the individual's eye gaze is towards the pre-assigned spatial region-of-interest.

17. An eye gaze tracking system of claim 16 further comprising a holder configured to position the image capturing device at a predetermined position in front of the individual, wherein the holder further comprises a cap configured to fit the individual's head.

18. An eye gaze tracking system of claim 15 further comprising a non-transitory computer-readable storage medium storing one or more programs, the one or more programs comprising instructions, which when executed by one or more processors of an electronic device, cause the electronic device to identify an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element; and
wherein the programs further comprise instructions, which when executed by one or more processors of the electronic device, cause the electronic device to:
obtaining one or more head pose parameters with regard to individual's head pose based on the head pose comparison step,
wherein the one or more head pose parameters comprise a total time for which the individual's head pose exceeds the threshold, and
wherein the one or more head pose parameters are (a) a marker of pre-screening, detecting or monitoring a developmental, cognitive, social, or mental disability or ability or (b) used to generate a gamification element.

19. An eye gaze tracking system of claim 18 further comprising a holder configured to position the image capturing device at a predetermined position in front of the individual, wherein the holder further comprises a cap configured to fit the individual's head.

20. An eye gaze tracking system of claim 15 further comprising a non-transitory computer-readable storage medium storing one or more programs, the one or more programs comprising instructions, which when executed by one or more processors of an electronic device, cause the electronic device to identify an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element; and
wherein the programs further comprise instructions, which when executed by one or more processors of the electronic device, cause the electronic device to:
calculating the percentage of total gazing time corresponding to at least one of the spatial region-of-interest,
calculating the percentage of total time for which the individual's head pose exceeds the threshold,
wherein each percentage of total gazing time and the percentage of total time that the head pose exceeds the threshold are (a) a marker of pre-screening, detecting or monitoring a developmental, cognitive, social, or mental disability or ability or (b) used to generate a gamification element.

21. An eye gaze tracking system of claim 20 further comprising a holder configured to position the image capturing device at a predetermined position in front of the individual, wherein the holder further comprises a cap configured to fit the individual's head.

22. An eye gaze tracking system of claim 15 further comprising a non-transitory computer-readable storage medium storing one or more programs, the one or more programs comprising instructions, which when executed by one or more processors of an electronic device, cause the electronic device to identify an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element; and
wherein the programs further comprise instructions, which when executed by one or more processors of the electronic device, cause the electronic device to:
calculating the percentage of total gazing time corresponding to at least one of the spatial region-of-interest,
calculating the percentage of total time for which the individual's head pose exceeds the threshold,
wherein each percentage of total gazing time and the percentage of total time that the head pose exceeds the threshold are (a) a marker of pre-screening, detecting or monitoring a developmental, cognitive, social, or mental disability or ability or (b) used to generate a gamification element,
wherein each unique spatial region-of-interest represents a quadrant of the visual stimulus, the head pose threshold range is about −15 to 15 degrees of yaw, about −15 to 15 degrees of pitch or a combination thereof, and
wherein the point-of-interest is selected from a group consisting of an eye in the visual stimulus, a mouth in the visual stimulus, interested object in the visual stimulus, non-interested object in the visual stimulus and a combination thereof.

23. An eye gaze tracking system of claim 22 further comprising a holder configured to position the image capturing device at a predetermined position in front of the individual, wherein the holder further comprises a cap configured to fit the individual's head.

24. An eye gaze tracking system of claim 15 further comprising a non-transitory computer-readable storage medium storing one or more programs, the one or more programs comprising instructions, which when executed by one or more processors of an electronic device, cause the electronic device to identify an individual's gaze pattern in response to a visual stimulus comprising the steps of:
detecting a head and at least one eye region of the individual;
obtaining a head pose of the individual;
comparing the obtained head pose with a predetermined threshold range and if the obtained head pose falls within the threshold range, selecting an eye gaze direction of the individual associated with such head pose;
obtaining one or more eye gaze parameters with regard to individual's eye gaze towards at least one point-of-interest in the visual stimulus using the selected eye gaze direction based on the head pose comparison step,
wherein the one or more eye gaze parameters are (a) a marker of pre-screening, detecting or monitoring a developmental, cognitive, social, or mental disability or ability or (b) used to generate a gamification element; and
wherein the programs further comprise instructions, which when executed by one or more processors of the electronic device, cause the electronic device to:
calibrate an image capturing device to the individual, wherein the calibration step further comprises the steps of
determine if the distance between the individual and the image capturing device is within first predetermined distance range;
in accordance with a determination that the distance not within the predetermined distance range, prompting to adjust the distance;
determine if a head orientation of the individual is within a predetermined orientation range;
in accordance with a determination that the head orientation not within the predetermined orientation range, prompting to adjust the head orientation; and
display the visual stimulus on a display unit after the calibration step.

25. An eye gaze tracking system of claim 24 further comprising a holder configured to position the image capturing device at a predetermined position in front of the individual, wherein the holder further comprises a cap configured to fit the individual's head.
